(19) United States
(12) Patent Application Publication
(10) Pub. No.: US 2021/0246502 A1
(43) Pub. Date: Aug. 12, 2021

(54) RAPID IDENTIFICATION OF STRAINS FROM SEQUENCE DATA

(71) Applicant: PRESIDENT AND FELLOWS OF HARVARD, Cambridge, MA (US)

(72) Inventors: William HANAGE, Cambridge, MA (US); Karel BRINDA, Brighton, MA (US); Michael Hartmann BAYM, Cambridge, MA (US)

(73) Assignee: PRESIDENT AND FELLOWS OF HARVARD COLLEGE, Cambridge, MA (US)

(21) Appl. No.: 17/251,343

(22) PCT Filed: Jun. 12, 2019
(86) PCT No.: PCT/US2019/036825

§ 371 Date: Dec. 11, 2020

Related U.S. Application Data
(60) Provisional application No. 62/684,134, filed on Jun. 12, 2018.

(51) Int. Cl. C12Q 1/6869; G16B 10/00

(52) U.S. Cl. CPC C12Q 1/6869; G16B 20/00; G16B 10/00

(57) ABSTRACT

Surveillance of circulating drug resistant bacteria is essential for healthcare providers to deliver effective empiric antibiotic therapy. However, molecular epidemiology does not occur on a timescale that is optimal for guiding patient treatment. Here the Inventors present a method called neighbor typing for inferring characteristics of an unknown bacterial sample by identifying the its closest relative in a database of known genomes. The Inventors demonstrate an implementation of this principle using sequence k-mer content, to identify both the closest relative and a phenotype of interest, in this case drug resistance. The Inventors show for the examples of S. pneumoniae and N. gonorrhoeae that this technique can be applied to data from an Oxford Nanopore device in real time and is capable of identifying the presence of a known resistant strain in 5 minutes of sequencing and 4 hours from sample collection, even from a clinical metagenomic sample. This flexible approach has wide application to pathogen surveillance and may be used to greatly accelerate diagnoses of resistant infections.

### STATEMENT REGARDING FEDERALLY SPONSORED RESEARCH OR DEVELOPMENT

This invention was made with government support under Grant No. AI106786 awarded by National Institutes of Health. The government has certain rights in the invention.

### FIELD OF THE INVENTION

The described technology relates to systems and methods for rapidly identifying strains from nucleic acid sequence information.

### BACKGROUND

In the study of antibiotic resistance, one can expend substantial resources in determining the properties of resistant strains, and surveillance is essential for healthcare providers to develop empiric and effective prescribing practices. However, the results of surveillance are typically not available on a timescale where they could inform treatment of individual patients. Here the Inventors present a method for matching data from an Oxford Nanopore device, as it is generated, with a database of known genomes to detect the closest match. This approach, which the Inventors term “lineage calling”, is capable of identifying the presence of a known resistant strain in 5 minutes, even from a complex metagenomic sample. This flexible, easily generalizable approach has wide application in surveillance, and by leveraging the presence of sequence variation across the genome that is linked to the resistance phenotype, may be used to greatly accelerate diagnoses of resistant infections.

### BRIEF DESCRIPTION OF THE FIGURES

FIG. 1: Overview of the RASE approach. The RASE approach uses three components: the RASE database, an approximate k-mer-based matching component based on ProPhyle, and a prediction component interpreting the risk based on the resistance of strains of the assigned phylogroup. In the load step, the precomputed RASE database is loaded into memory. The RASE pipeline iterates over reads streamed from the nanopore sequencer. Each read is matched against the database using ProPhyle. Retrieved assignments are propagated to the leaves and similarity scores computed. These are used to identify best-matching strains (possibly many) and to update weights associated with these strains. Indeed, a single read is rarely specific, it typically matches equally scored multiple nodes. The best phylogroup is identified and a phylogroup score calculated (PGS). Based on the resistance profiles of strains in this phylogroup, susceptibility to each of the antibiotics is predicted from the best match and reported together with a susceptibility score quantifying the risk of resistance.

FIG. 2: Timeline and rank plots for an isolate. Aa) Number of reads, phylogroup score, and susceptibility scores for individual antibiotics as a function of time from the start of sequencing. The point markers depict the times of stabilization for the predicted phylogroup, the alternative phylogroup and the most similar isolate, respectively. Ab), Ac), and Ad) Similarity rank plots for selected time points (1 minute, 5 minutes, and the end of sequencing). The bars correspond to 70 best matching isolates in the database and display the predicted level of sample-to-strain relative similarity (i.e., normalized weights). They are arranged by rank and colored according to the presence in the predicted, alternative or another phylogroup. The bottom panels display the susceptibility profiles of the isolates. Timeline and rank plots for a metagenome. The figure is of the same format with Ba), Bb), Bc), and Bd).

FIG. 3. A) Prevalence of resistance phenotypes across phylogroups. Statistics on prevalence of resistance phenotypes across phylogroups before and after the ancestral state reconstruction step. B) Setting k-mer length for S. pneumoniae. K-mer length in RASE is set based on the kmer complexity of the genome, i.e., the number of different substrings of length k as a function of k. The RASE strategy is to use shortest discriminative k-mers so that regions between

sequencing errors get covered by sufficiently many k-mers and the k-mers are still discriminative.

FIG. 4: Size and memory footprint of the RASE database and index. The graph compares the size of the ProPhyle RASE index to the size of the original sequences: original draft assemblies (seq-fa), original draft assemblies compressed using gzip (seq-fagz), memory footprint of ProPhyle with the RASE index (ind-mem), and size of the ProPhyle RASE index compressed for transmission (ind-transm).

FIG. 5: Timeline of resistance genes. Number of occurrences of individual resistance genes in reads of SP02, as a function of time for the first hour of nanopore sequencing.

FIG. 6: MIC intervals for individual isolates in the RASE database. The plot illustrates MIC intervals and point values extracted from. Each panel corresponds to a single antibiotic, while vertical lines and points correspond to individual isolates. Their colors correspond to the resistance category after applying a breakpoint (horizontal lines). When a resistance category could not be assigned directly (i.e., in case of an interval crossing the breakpoint line), then it was inferred using ancestral state reconstruction.

FIG. 7: Ancestral state reconstruction of resistance categories in the RASE database. Each panel corresponds to a single antibiotic and displays the database phylogenetic tree, colored according to the reconstructed resistance categories for the antibiotic (blue, green, red, violet correspond to ‘susceptible’, ‘unknown—inferred susceptible’, ‘non-susceptible’, ‘unknown—inferred non-susceptible’, respectively).

FIG. 8: Subword complexity of pneumococcus. The plot depicts the number of canonical k-mers as a function of k for S. pneumoniae ATCC 700669 (NC_011900.1) and for a random DNA text containing all possible k-mers. For k<10, the pneumococcus k-mer composition is similar to the one of random text. Fork >14, the k-mer sets are almost saturated and the complexity grows very slowly. Since the genome has a finite length and is circular, the function has an asymptote, which would be attained for k equal to the length of the genome (2,221,315). The highlighted region corresponds to the range of k values, which are suitable for use in RASE.

FIG. 9: Delays in prediction based on the k-mer length. The plot displays delays in prediction as a function of the used k-mer length, for all experiments and all possible k-mer lengths. Each horizontal panel displays times required for stabilization of one of the three predictions: phylogroup (PG), alternative phylogroup (PG2), and closest isolate (Isolate). Every column within a panel corresponds to a single k-mer length. When the required time exceeded 1 hour, the point is displayed at the top. Experiments where phylogroup could not be identified are plotted in red. The highlighted column corresponds to the k-mer length used for constructing RASE.

FIG. 10: Cumulative proportion of matching k-mers as a function of time. This figure shows that nanopore devices provide the data shortly after the start of sequencing and then the quality drops down.

FIG. 11: Proportions of matching k-mers for isolates from the NCTC collection (the two files correspond to the pneumococcal RASE databases, only first 60 species displayed). The figure shows that if we use a wrong db (e.g., we use the pneumococcal db, but sequence Enterococcus faecalis), we can recognize that from the proportion of matching k-mers.

FIG. 12: Proportions of matching k-mers for isolates from the NCTC collection (the two files correspond to the gonococcal RASE databases, only first 60 species displayed). The figure shows that if we use a wrong db (e.g., we use the pneumococcal db, but sequence Enterococcus faecalis), we can recognize that from the proportion of matching k-mers.

FIG. 13: Predicted phenotypes. of S. pneumoniae for a) database isolates, b) non-database isolates, and c) metagenomes. The figure displays actual and predicted resistance phenotypes (S=susceptible, R=nonsusceptible) for individual experiments, as well as information on match of the predicted sequence type and clonal complex. Resistance categories in bold were inferred using ancestral reconstruction and were also confirmed using phenotypic testing. Metagenomic samples are sorted by the estimated proportion of S. pneumoniae reads.

FIG. 14: Predicted phenotypes of N. gonorrhoeae for a) database isolates and b) clinical isolates. The figure is in the same format as FIG. 14.

FIG. 15: Predicted phenotypes. The table displays actual and predicted resistance phenotypes (S=susceptible, R=non-susceptible) for individual experiments, as well as information on match of the predicted sequence type and clonal complex

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

Infections pose multiple challenges to healthcare systems, contributing to higher mortality, morbidity, and escalating cost. Clinicians must regularly make rapid decisions on empiric treatment without knowing if a patient's clinical syndrome is due to a drug resistant organism. In some cases, this is directly linked to poor outcomes; in the case of septic shock, the risk of death increases by an estimated 10% with every 60 minutes delay in initiating effective treatment.

The molecular epidemiology of infectious disease allows us to identify high-risk pathogens and determine their patterns of spread, on the basis of their genetics or (increasingly) genomics.

Conventionally such studies have been conducted in retrospect, as outbreak investigations or the identification of newly emerged strains after the fact, but this has been changing with the availability of new and increasingly inexpensive sequencing technologies. For example, the Centers for Disease Control used to sequence a fraction of the influenza strains they collected, on the basis of whether their phenotype suggested they should be further characterized.

However, since 2015 this has been inverted with the “Sequence First” pipeline, in which the genome is determined for all influenza isolates as soon as possible, and made publicly available. The benefit of this approach is both that it generates and publishes sequence data quickly and it is efficient. Indeed, an isolate that is closely related to something already sampled is likely to share phenotypic properties with it, for example, antibiotic resistance.

The clinical question of whether an antibiotic is likely to work, i.e. the pathogen is susceptible, is not equivalent to identifying whether a pathogen carries those mutations or genes that are known to confer resistance. Prescription has long been informed by correlative features when causative ones are difficult to measure, for example whether the same syndrome or pathogen occurring in other patients from the same clinical environment have responded to a particular antibiotic. This also has been observed at the genetic level as well, as a result of genetic linkage between resistance elements and the rest of the genome. An example is given by the pneumococcus (Streptococcus pneumoniae). The Centers for Disease Control have rated the threat level of drug resistant pneumococcus as ‘serious’. While resistance arises in pneumococci through a variety of mechanisms and genes, approximately 90% of the variance in the minimal inhibitory concentration (MIC) for antibiotics of different classes can be explained by the loci determining the strain type alone, even though none of the loci used for strain classification themselves causes resistance. Thus, in the overwhelming majority of cases, resistance can be inferred from coarse strain typing based on population structure. This population structure could be leveraged to offer an alternative approach to detecting resistance in which rather than detecting high-risk genes, the Inventors identify high-risk isolates.

In this paper, the Inventors introduce a method which can bring molecular epidemiology closer to the bedside and provide information relevant to treatment at a much earlier stage in the process. Sequence generated in ‘real time’ can be matched to a database of genomes to identify the closest relative. Because closely related isolates in most cases have similar properties, this yields an informed “first guess” of the pathogen's phenotype. The Inventors demonstrate this for Streptococcus pneumoniae (the pneumococcus) and Neisseria gonorrhoeae (the gonococcus), specifically for the identification of drug resistant clones and show that the Inventors can make predictions within minutes, as the sequencer is running using Oxford Nanopore Technology. The method has many potential applications, depending on the specific pathogen and quality of the databases available for matching, which the Inventors discuss together with its limitations.

The problem of antibiotic resistance poses multiple challenges to healthcare systems. Clinicians must make rapid decisions on appropriate treatment in the absence of data on whether the patient is suffering disease due to a drug resistant organism. In some cases, this is directly linked to poor outcomes; in the case of sepsis it is estimated that every 60 minutes delay in effective treatment increases the risk of death by approximately 10%. [needs citation]. Drug resistant infections hence contribute to higher mortality, morbidity and the escalating cost of healthcare. The problem has been described in apocalyptic terms.

There is hence great interest in developing rapid ways to detect the presence of a resistant strain in a sample, for purposes of diagnostics and surveillance, with a particular focus on the use of genomics. In principle, if a resistance gene or mutation can be detected in a sample, this could be sufficient to inform prescribing. For this to be viable, several conditions must be satisfied: foremost, the resistance determinant must be already known such that the Inventors can test for it, it must also be sufficiently different from susceptible variants to be readily detected. The genomic context is also important, as loci with homology to known resistance determinants are also found in non-pathogens. As the ideal is to sequence as directly as possible from clinical samples, with minimal culture steps. This implies a metagenomic sample containing sequence from many different taxa, and the genomic context of the resistance locus may be obscured if the Inventors use short read technologies for sequencing. Similar problems present themselves in the use of PCR to specifically amplify resistance genes, namely the Inventors would need to know what sequence the Inventors are looking for, and the Inventors would not be able to determine the genomic context from a positive result, merely that the gene was present. An ideal approach will also be deployable close to the point of care, and in resource poor settings.

One of the features of drug resistant loci is that horizontal gene transfer can import them into multiple genetic backgrounds. However, it is not true that all genetic backgrounds are equally likely to contain resistance genes. It has long been known that some clones are more likely to be resistant, to the extent that in some cases expert committees collect data to characterize them and name them. The pneumococcus (Streptococcus pneumoniae) is a major pathogen, responsible for approximately 1.6 million deaths per annum, and the Centers for Disease Control have rated the threat level of drug resistant pneumococcus as “serious”. The Pneumococcal molecular epidemiology network has named 43 clones, with their associated resistance characteristics and serotypes. These PMEN clones can be characterized by Multi Locus Sequence Typing (MLST) to fall into a minority of the many circulating pneumococcal clones. MLST assays variation at seven regions around the genome to define the sequence type or ST. Importantly, none of the regions sequences in MLST cause resistance, however a GWAS analysis of which variation was causing resistant in the pneumococcus found that as much as 90% or more of the variance in the minimal inhibitory concentration (MIC) for multiple antibiotics of different classes, could be explained by the MLST data4. While none of the MLST loci themselves cause resistance, they are sufficiently closely linked with it that they produce confounding population structure.

This population structure can be leveraged to offer an alternative approach to detecting resistance in which rather than detecting high-risk genes, the Inventors identify high-risk lineages. The additional information available from genomic data allows a better definition of those closely related parts of the population associated with resistance or susceptibility, over and above the STs and clonal complexes5-8 that are defined by MLST, and the Inventors call these “phylogroups”. High-risk phylogroups can be readily determined by analysis of existing high-quality draft genomes generated with short reads, together with suitable metadata on MICs. The Inventors then compare the sequence under test with this in order to define the phylogroup and any associated properties, such as drug resistance. The approach removes the requirement that resistance loci be known in advance, as the Inventors are not attempting to identify genetic variation that causes resistance, but variation that is associated with it. While in principle the phylogroup could be detected from short read data, a more attractive option is to use long read data such as that produced by Oxford Nanopore Technology (ONT). Although ONT has a very high (˜10%) per base error rate, it is highly portable and deployable in field conditions9, and furthermore sequencing reads are provided in a stream so the results can be reported real-time and sequencing stopped at any point, as soon as enough information is collected. Recently, ONT has been shown to provide rapid re-identification of human samples within minutes or predict antibiotic resistance in Mycobacterium tuberculosis within the same day.

Here the Inventors present methods to match a sample against a database of known genomes, from isolates for which resistance has already been determined, and predict resistance based on the antibiograms of the best matches. The Inventors demonstrate using the example of pneumococcus and five antibiotics (penicillin, ceftriaxone, trimethoprim, erythromycin, and tetracycline) that the Inventors can identify known resistant clones, and their serotype, on a standard laptop within minutes. The Inventors' solution is suitable for applications even in resource-poor countries, making it not only useful for diagnosing infections, but also enhancing surveillance.

Described herein is a method of classifying properties of one or more biological strains in a sample, including providing a biological sample including one or more biological strains, sequencing DNA in the biological sample, comparing one or more phylogroups to DNA sequences of at least two loci in the biological sample, wherein the phylogroup is associated with one or more properties, and classifying the one or more biological strains into the one or more phylogroups, thereby classifying properties of biological strains in the sample. In other embodiments, comparing one or more phylogroups to DNA includes at least two, at least five, at least 10-50, 50-100, 100-200, 200-500, 500 or more loci. In other embodiments, the biological sample is metagenomic. In other embodiments, the sequencing method has up to 20% error. In other embodiments, the sequencing methods has up to 1, 2, 3, 4, 5, 6, 7, 8, 9, 10, 11, 12, 13, 14, 15, 16, 17, 18, 19 or more % error. In other embodiments, the sequencing method provides data in a real-time stream. In other embodiments, the one or more phylogroups includes an index of 13-45 nucleotide sequences in length. In other embodiments, the one or more phylogroups includes an index of nucleotide sequences, each of at least 15 nucleotides in length. In other embodiments, the nucleotide sequences are each 18 nucleotides. In other embodiments, the one or more biological strains into the one or more phylogroups includes weighted scoring of the sequences of the at least two loci. In other embodiments, the weighted scoring includes higher weighting for longer sequences and/or sequences covering multiple accessory genes. In various embodiments, this can include a phylogenetic tree, with k-mer sets in the leaves, weighted scoring including an index value based on maximum sequence length and discounted proportionally to zero at the specified minimal sequence length, an index value based on an index value of zero or nominal amount for core genome, and proportionally or exponentially increased for one or more accessory genes. In other embodiments, the sequences are at least 200, 300, 400, 500, 600, 700, 800, 900 or more nucleotides. In other embodiments, the sequences are at least 1000 nucleotides. One of skill in the art understands accessory genes to be genes flexibly expressed across biological strains of a species, in contrast to core genome which is expressed across all biological strains in a species. In other embodiments, the one or more properties comprise one or more of: antibiotic resistance, pathogenicity, and serotype. In other embodiments, the one or more biological strains are bacteria. In other embodiments, the bacteria comprise pneumococcus. Other examples include streptococcus, pseudomonas, salmonella, e. coli, among others. In other embodiments, the one or more biological strains are virus. In other embodiments, the one or more biological strains are fungi.

Further described herein method of therapeutic selection, including providing a biological sample isolated from a subject, wherein the biological sample includes one or more biological strains, sequencing DNA in the biological sample, comparing one or more phylogroups to DNA sequences of at least two loci in the biological sample, wherein the phylogroup is associated with one or more properties, classifying the one or more biological strains into the one or more phylogroups, thereby classifying properties of biological strains in the sample, selecting a therapeutic agent based on the properties of biological strains in the subject, and administering the therapeutic agent to the subject. In other embodiments, the biological sample is metagenomic. In other embodiments, the one or more phylogroups includes an index of nucleotide sequences, each of at least 15 nucleotides in length, wherein classifying the one or more biological strains into the one or more phylogroups includes weighted scoring of the sequences of the at least two loci, and further wherein weighted scoring includes higher weighting for longer sequences and/or sequences covering multiple accessory genes. In other embodiments, the one or more biological strains are bacteria and the properties comprise antibiotic resistance. In other embodiments, wherein the method selecting a therapeutic agent includes choosing an antibiotic, wherein the one or more biological strains are susceptible to the antibiotic. In other embodiments, the bacteria comprise pneumococcus.

Additionally, described herein a method of rapid screening of a biological sample, providing a biological sample isolated from a subject, wherein the biological sample includes one or more biological strains, sequencing DNA in the biological sample, comparing one or more phylogroups to DNA sequences of the at least two loci in the biological sample, wherein the phylogroup is associated with one or more properties, and classifying the one or more biological strains into the one or more phylogroups, thereby classifying properties of biological strains in the sample, wherein sequencing has up to 20% error and provides data in a real-time stream, wherein the one or more phylogroups includes an index of nucleotide sequences, each of at least 15 nucleotides in length, wherein classifying the one or more biological strains into the one or more phylogroups includes weighted scoring of the sequences of the at least two loci with higher weighting for longer sequences and/or sequences covering multiple accessory genes, and further wherein the sequences are at least 1000 nucleotides. In other embodiments, the biological sample is metagenomic. In other embodiments, the rapid screening is less than 10 minutes. In other embodiments, the biological sample is substantially free of genomic DNA from the subject. In other embodiments, the biological sample consists essentially of DNA from one or more biological strains. In various embodiments, the biological sample is prepared by a method of removing human DNA. This includes for example, a blood spin and methylation pull-down. In various embodiments, rapid may include screening within 60 minutes or less minutes, 45 minutes or less, 30 minutes or less, 15 minutes or less, 10 minutes or less, 5 minutes or less from initiation of sequencing.

Further described herein is a method of diagnosis, including, obtaining a biological sample from a subject, sequencing DNA in the biological sample, comparing one or more phylogroups to DNA sequences of at least two loci in the biological sample, wherein the phylogroup is associated with one or more properties, classifying the one or more biological strains into the one or more phylogroups, and diagnosing the subject as infected with one or more biological strains based on phylogroup classification. In other embodiments, the sequencing method has up to 20% error and provides data in a real-time stream, wherein the one or more phylogroups includes an index of nucleotide sequences, each of at least 15 nucleotides in length, wherein classifying the one or more biological strains into the one or more phylogroups includes weighted scoring of the sequences of the at least two loci with higher weighting for longer sequences and/or sequences covering multiple accessory genes, and further wherein the sequences are at least 1000 nucleotides.

### Example 1

### Overview

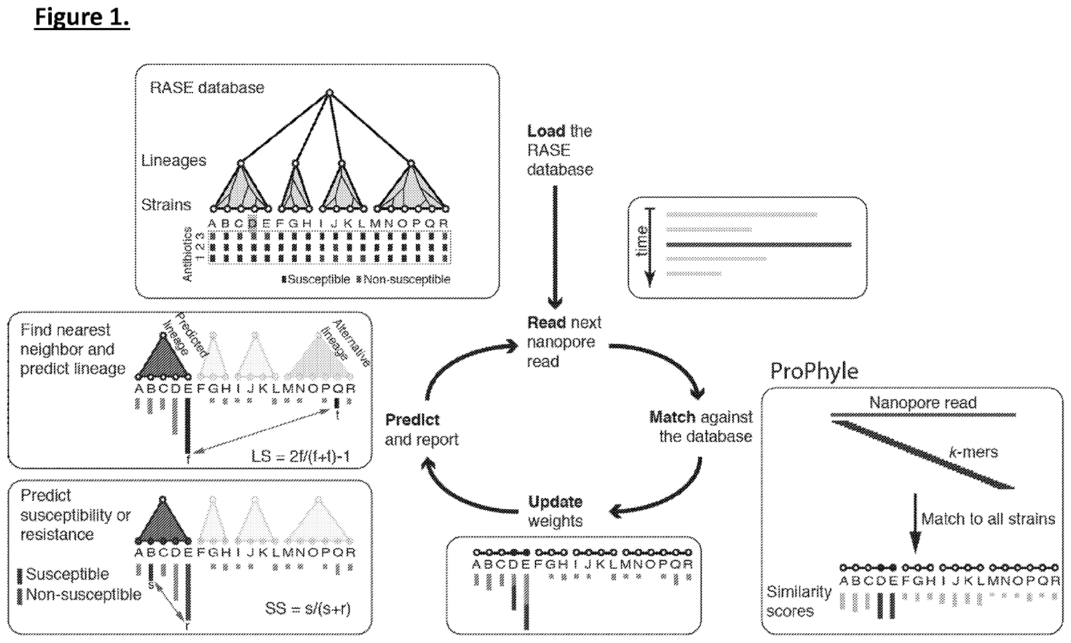

RASE uses rapid approximate k-mer-based matching of long sequencing reads against a database of genomes to predict resistance via lineage calling, using two key components: a database containing genomic data and associated antibiograms, and a prediction pipeline. The database contains a highly compressed lossless k-mer index, a representation of the tree population structure, and metadata such as a phylogroup, serotype, sequence type and resistance profiles (see “Resistance profiles”). The pipeline iterates over reads from the nanopore sequencer and provides real-time predictions of phylogroup and resistance (FIG. 1).

### Example 2

### Resistance Profiles

For all antibiotics, RASE associates individual isolates with a resistance category, susceptible or non-susceptible. First, MIC values are mined using regular expressions from the available textual antibiograms, i.e., strings describing an interval of possible MIC values. Second, the acquired intervals are compared to the antibiotic-specific breakpoints (see below). If a given breakpoint is above or below the interval, susceptibility or non-susceptibility is reported, respectively. However, no category can be assigned at this step if the breakpoint lies within the extracted interval, an antibiogram is entirely missing, or an antibiogram is present, but parsing failed. Third, missing categories are inferred using ancestral state reconstruction on the associated phylogenetic tree while maximizing parsimony (i.e., minimizing the number of nodes switching its resistance category).

The RASE database is constructed with the standard EUCAST breakpoints ([g/ml]): Benzylpenicillin (PEN): 0.06, Ceftriaxone (CRO): 0.25, Trimethoprim (TMP): 1.00, Erythromycin (ERY): 0.25, and Tetracycline (TET): 1.00. The breakpoints are set conservatively, i.e., non-susceptibility is preferred over susceptibility for intermediate values. While the Inventors have used the above values in the present work, others may be readily defined and the database rapidly updated. This is especially useful in the case where breakpoints may vary depending on the site of infection (as is the case with pneumococcal meningitis and otitis media, where lower MICs are considered to be resistant (REF)).

### Example 3

### K-Mer-Based Matching

RASE uses the ProPhyle classifier and its ProPhex component to identify the most similar genomes in the database for every sequencing read. Its index stores k-mers of all isolates' assemblies in a highly compressed form, reducing the required memory footprint. The database k-mers are first propagated along the phylogenetic tree and then greedily assembled to contigs. The obtained contigs are then placed into a single text file, for which a BWT-index is constructed. The index can be searched for individual k-mers, retrieving a list of nodes whose descending leaves correspond to isolates containing that k-mers.

In course of sequencing, every read is matched against the index and matches for all read's k-mers retrieved. These matches are then propagated to the level of leaves and isolates with the highest number of shared k-mers identified.

### Example 4

### Predicting Resistance from Phylogroups

All isolates in the database are associated with similarity weights, which are set to zero at the start of the run. Each time a new read is matched against the DB, the weights for the best match are increased according to the read's “information content”, calculated as the number of shared k-mers between a genome and the read, divided by the number of best hits.

Predictions are calculated based on the current state of the weights and the lineage or phylogroup in which the best-matched isolate is found. First, a phylogroup is predicted as the phylogroup of the best matching isolate. Then, a phylogroup score is calculated PGS=2f/(f+t)−1, where f and t denote the scores of the best matches in the first and second best phylogroup. If PGS is higher than a specified threshold (0.6 in default settings), the call is considered successful. If the score is lower than this, the read cannot be securely assigned to a phylogroup, and this counts as a failure. Reads that do not match are not used in subsequent analysis to predict resistance.

Resistance is predicted for individual antibiotics independently, using weights within the predicted phylogroup. While certain phylogroups are certainly associated with susceptibility, some others are not. For the latter the Inventors propose the use of the susceptibility scores which combine the resistance characteristics of the most similar strains in the RASE database. A susceptibility score is calculated as SUS=s/(s+r), where s and r denote the score of the best susceptible and non-susceptible strains within the phylogroup. If SUS is greater than a specified threshold (0.6), susceptibility to the antibiotic is reported, non-susceptibility otherwise.

### Example 5

### Lower Time Bounds on Resistance Gene Detection

Real-time classification is simulated from base-called nanopore reads. Timestamps of individual reads are first extracted and then used for sorting the reads. When the RASE pipeline is applied, the times of assignments are compared to the original timestamps to ensure that the prediction pipeline is not slower then sequencing. A complete genome assembly is computed from Nanopore reads using the CANU (version xxx, default parameters). Prior to the assembly step, reads are filtered: they must be at least 1000 bp long and must have at least 10% of matching

18-mers with some of the reference draft assemblies. The obtained assembly is further corrected by Pilon (version xxx, default parameters) using Illumina reads. Ariba [pmid: 29177089] is then applied to detect resistance genes present in this assembly (version xx, with default parameters).

The nanopore reads are mapped using Minimap2 to the cleaned assembly and their coordinates retrieved. To be considered informative reads must be long enough (>1000 bp) and they must fully contain the given resistance gene. Timestamps of the resistance-informative reads are extracted and associated with the genes.

### Example 6

### MinION Library Preparation

Cultures were grown in Todd-Hewitt medium with 0.5% yeast extract (THY; Becton Dickinson and Company, Sparks, Md.) at 37° C. in 5% CO2 for 24 hrs. High molecular weight (>1 ug) genomic DNA was extracted and purified from cultures using DNeasy Blood and Tissue kit (QIAGEN, Valencia Calif.). DNA concentration was measured using Qubit fluorometer (Invitrogen, Grand Island N.Y.). Library preparation was performed using the Oxford Nanopore Technologies 1D ligation sequencing kit SQK LSK108, R9 version, according to the manufacturer's instructions.

Sequencing was performed on the MinION MK1. Base-calling was performed using Metrichor simultaneously with sequencing. All reads passing Metrichor quality check were used in the further analysis.

### Example 7

### A Database of Resistant Elements

To predict resistance in isolates and clinical samples the Inventors built a database of Resistance Associated Sequence Elements (RASE). The Inventors generated a k-mer-based representation of lineages that the Inventors can then use to predict resistance using approximate matching. Following an analysis of the S. pneumoniae genome and characteristics of ONP reads, the Inventors set k=18 (see Methods). The Inventors' method depends on the initial availability of good quality data. The Inventors developed an extensive review of the published literature using a bespoke tool (MetaMedA) to identify appropriate papers. The results, including extracted textual supplementary tables, are available on http://github.com/c2-d2/pneumo-data.

The Inventors eventually chose genomes of pneumococci sampled from a carriage study in Massachusetts children as the main reference dataset; it consists of 616 carriage samples isolated from Massachusetts children and comprises excellent quality resistance data, together with high quality draft genome assemblies from Illumina reads. Based on the measured MIC, the Inventors assigned each isolate to an antibiotic-specific resistance category using standard breakpoints (see Methods). Ancestral state reconstruction was used to infer categories for cases where exact MICs were not recorded. Out of all 616 isolates, the Inventors obtained 341, 485, 480, 484 and 551 isolates susceptible to penicillin, ceftriaxone, trimethoprim, erythromycin, and tetracycline, respectively.

### Example 8

### Lineage Calling Using Inexact Matching

The Inventors have developed an approach the Inventors call “lineage calling” (FIG. 1) to accurately match a nanopore read to the phylogroup from which it came—where phylogroup as described above is a clade associated with either resistance or susceptibility. Lineage calling has several advantages, but the major one is time, as it allows us to leverage the real-time nature of nanopore sequencing provided the Inventors can assign them to a lineage rapidly enough. For lineage calling was done using a modified version of ProPhyle; an accurate, resource-frugal and deterministic phylogeny-based DNA classification tool using the Burrows-Wheeler Transform, which can assign nanopore reads to phylogenetic trees on a standard laptop. For the Inventors' dataset, consisting of a phylogenetic tree and k-mer sets in the leaves, the Inventors constructed a lossless ProPhyle k-mer index. Generally speaking, longer and more specific reads, such as those covering multiple accessory genes, tend to have high scores; whereas short and non-specific reads, such as the ones from the core genome, have low scores. Cumulative scores are then used to measure how similar a sample is to known genomes associated with resistance, already in the database.

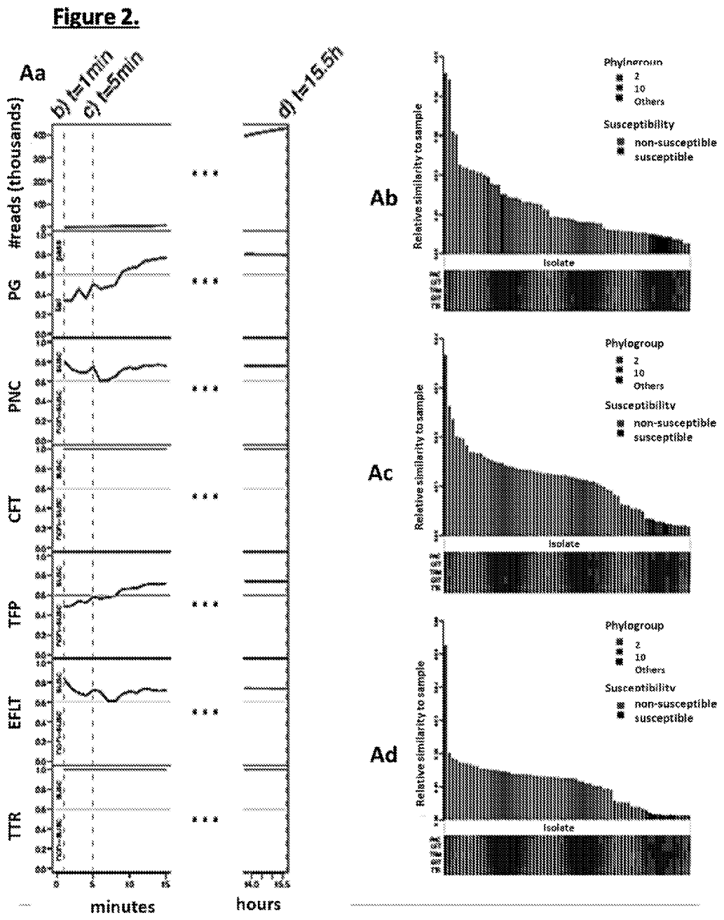

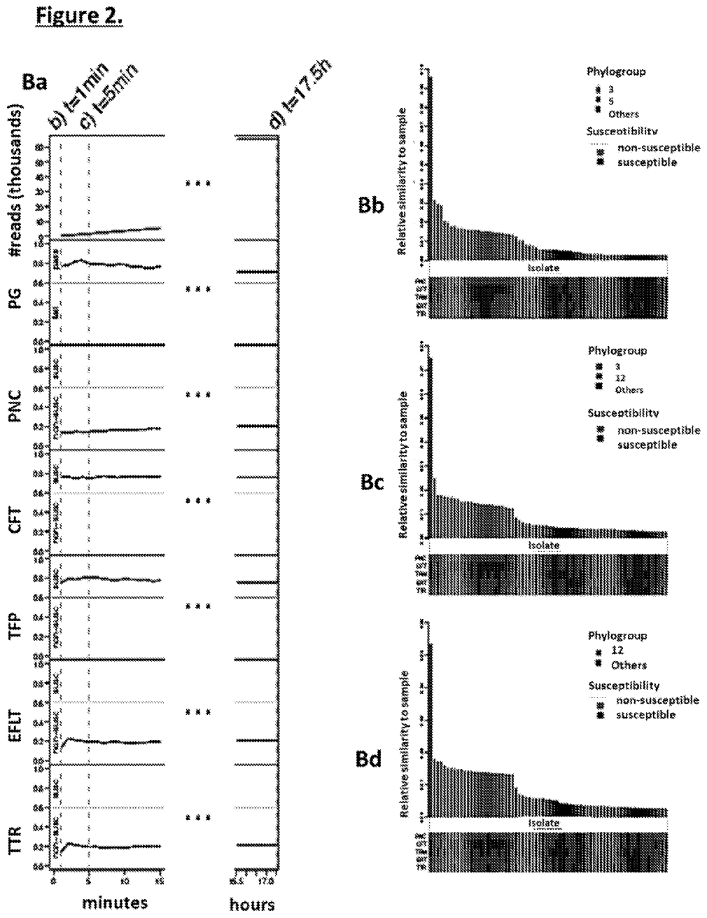

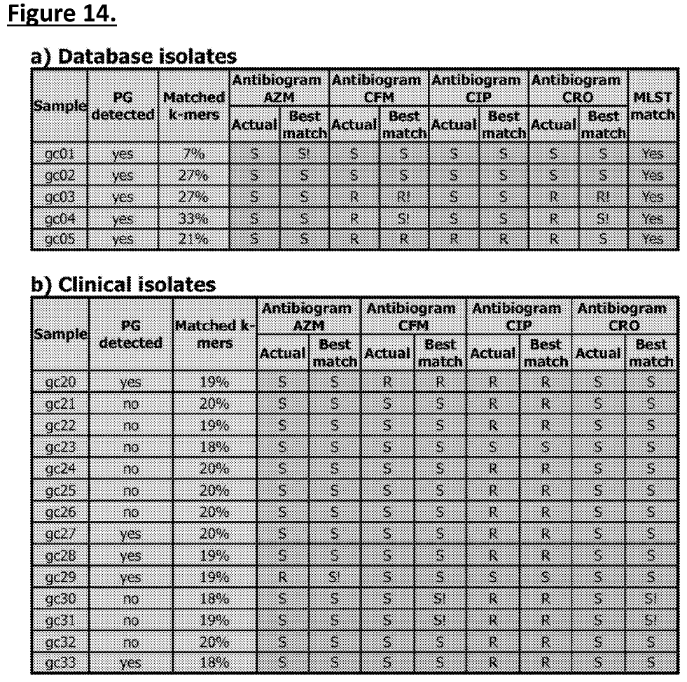

The results of two example RASE profiles are shown in FIG. 2, as bar charts plotting the matches in order of rank from best to worst. Results are shown after 5 minutes of running the sample on ONT, with concomitant matching to the RASE database using ProPhyle. The true lineage and resistance phenotype of all samples, together with those inferred through lineage calling are shown in FIG. 14. FIG. 2A shows proof of principle; this is the profile obtained from a fully susceptible isolate, with serotype 11D and identified as ST 62 by MLST. This isolate was among those used to build the RASE database, and so this tests whether the high error rate of ONT sequencing will hinder the Inventors' approach. In fact, the correct phylogroup is assigned within 5 minutes, and the best match is the actual isolate used in the test. Note that due to errors in the sequence from the ONT device, only 20% of the bases matched to k-mers in the RASE database, but this was sufficient.

To investigate samples not present in the RASE database, the Inventors examined four isolates for which the antibiogram and serotype were known, but the genome had not been sequenced and the lineage was unknown. The results are summarized in FIG. 14. The Inventors compare three characteristics of the sample to assess the Inventors' performance: the serotype, the sequence type (ST) and the antibiograms (penicillin, ceftriaxone, trimethoprim, erythromycin, and tetracycline resistance according to NCLSI breakpoints). ST is the gold standard for strain assignment by MLST and divides the pathogen population into clonal complexes (equivalent to lineage). In all cases the correct clonal complex is identified, even if the correct ST is absent from the RASE database, indicating the strength of the lineage calling method in rapidly detecting similarity. These are each cases of known PMEN clones, with characteristics shown in FIG. 14. Again, these results were available within five minutes of starting Nanopore sequencing.

Because culture introduces significant delays, metagenomic samples containing DNA directly isolated from a clinical sample would be preferable. FIG. 2B shows the results of analysis of ONT sequence from of a metagenomic sample, obtained from sputum of a patient suffering from ventilator-associated pneumonia. DNA was prepared and sequencing carried out with pretreatment to reduce the proportion of human DNA. The sample contains DNA from multiple bacterial species, and as a result few of the reads match to the k-mers in the RASE database (7% in contrast with 20% for the first sample described above).

Nevertheless, the Inventors were able to identify the presence of the Swedish 15A clone (ST63) which is also known to be associated with other resistance phenotypes including macrolides and tetracyclines. This isolate was confirmed to be resistant to the macrolides clindamycin and erythromycin, as well as tetracycline and oxacillin (FIG. 14).

### Discussion

Effective methods for detecting resistance from gene sequence do not need to perform GWAS in reverse—there is no requirement to detect the variation that causes the phenotype, only that it be sufficiently strongly associated with the phenotype to make reliable predictions. The three experiments presented here show that where an identical genome is present, ProPhyle accurately matches it in five minutes, and where the genome is not present the closest relative is matched to in a similar time span. Moreover, ProPhyle can be used successfully with metagenomic data, here identifying the presence of the Sweden 15A-23 clone in a sputum sample taken from a patient with VAP. Together, these results suggest that the Inventors can achieve robust lineage calling, even from complex data, minutes after the ONT device starts running.

This approach is not limited by the relatively high error rate of ONT because it is not attempting to define the exact genome sequence of the sample under test, but merely which lineage it represents. As a result, even when a small fraction of k-mers in the read are informative in matching to the RASE database, this is sufficient to call the lineage. This has the benefit of being faster than gene detection by virtue of the informative k-mers being distributed throughout the genome, and so more likely to appear in the initial reads from the nanopore. Therefore, the approach the Inventors present here can be seen as an application of compressed sensing: by measuring a sparse signal distributed broadly across the Inventors' data the Inventors can identify it with comparatively few error-tolerant measurements.

These results suggest a two-step model for determining resistance, in which the first is to characterize the population with highly accurate, high quality draft genomes and excellent quality metadata, and the subsequent analysis of a sample using ONT and the RASE software. Public health laboratories are increasingly collecting datasets suitable for use with RASE. The Centers for Disease Control have started using WGS to characterize samples from their Active Bacterial Core Surveillance system, which contains isolates and MIC data from all isolates of S. pneumoniae causing invasive disease in a population of more than 23 million. As a result of this initiative, genome sequences for 2316 isolates collected from 2015 are already. It is not impossible that an infection could be caused by a lineage not present in this sample, but it is unlikely. In the event that the sequenced isolate belongs to a clade that is absent from the database, RASE reports comparable similarity for multiple different sequence clusters and the cluster assignment confidence drops accordingly (see supplementary online material).

A more serious issue, which the Inventors have not encountered in this study, but which may limit the application of the Inventors' approach to other pathogen-drug combinations, is the degree of linkage between resistance and a specific lineage. If this is sufficiently low, such that there is very weak association between lineage and resistance phenotype, then the Inventors would not expect this approach to be useful. This is particularly the case if resistance can arise from a single mutation during the course of treatment (such as porin mutations which confer diminished susceptibility to carbapenems). Such an eventuality will not be detectable by any sequence based method, and will mislead conventional gold standard susceptibility testing if the mutation has not already arisen.

In terms of time the major limitation of this approach is the time required for sample preparation, which here includes DNA isolation and library preparation. However, the Inventors note that the Voltrax technology already allows genomic DNA to be supplied to ONT, removing the need for library prep. So the limiting time is that which is required for the isolation of DNA and library prep; approximately 2 hours altogether using the ligation library method applied in this work. It should be noted that this has been further reduced, with a Rapid Sequencing Kit offering library preparation in ten minutes (https://www.protocols.io/view/ultra-long-read-sequencing-protocol-for-rad004-mrxc57n). Further advances in this space, including reduced costs, will be required to bring the method closer to the bedside.

The benefits of lineage calling are in identifying high-risk clones earlier. It is easy to see how the Inventors' approach may be extended to include calling specific resistance loci, where they are known, but it is not limited by the requirement to know them in advance. In fact lineage calling can be used to detect any phenotype that is sufficiently tightly linked to a phylogeny, to identify for instance highly virulent strains that might merit closer attention. Further applications may include rapid outbreak investigations, as the closely related isolates involved in the outbreak will all be predicted to match to the same strain in the RASE database. The approach also lends itself to enhanced surveillance, including work in field situations—for example the recent Ebola outbreak in West Africa, saw ONT devices used in remote locations without centralized and advance healthcare facilities. Finally, this approach is not at present intended to supplant empiric therapies. Given the urgency of instituting appropriate therapies, prescriptions should be made as early as possible. However, the Inventors may be able, through lineage calling of samples taken when the tentative diagnosis is made, to make great improvements in response time when the initial therapy is inadequate.

### Example 9

### Overview

RASE uses rapid approximate k-mer-based matching of long sequencing reads against a database of genomes to predict resistance via neighbor typing, using two key components: a database containing genomic data and associated antibiograms, and a prediction pipeline. The database contains a highly compressed exact k-mer index, a representation of the tree population structure, and metadata such as a lineage, serotype, sequence type and resistance profiles (see ‘Resistance profiles’). The pipeline iterates over reads from the nanopore sequencer and provides real-time predictions of lineage and resistance (FIG. 1).

### Example 10

### Resistance Profiles

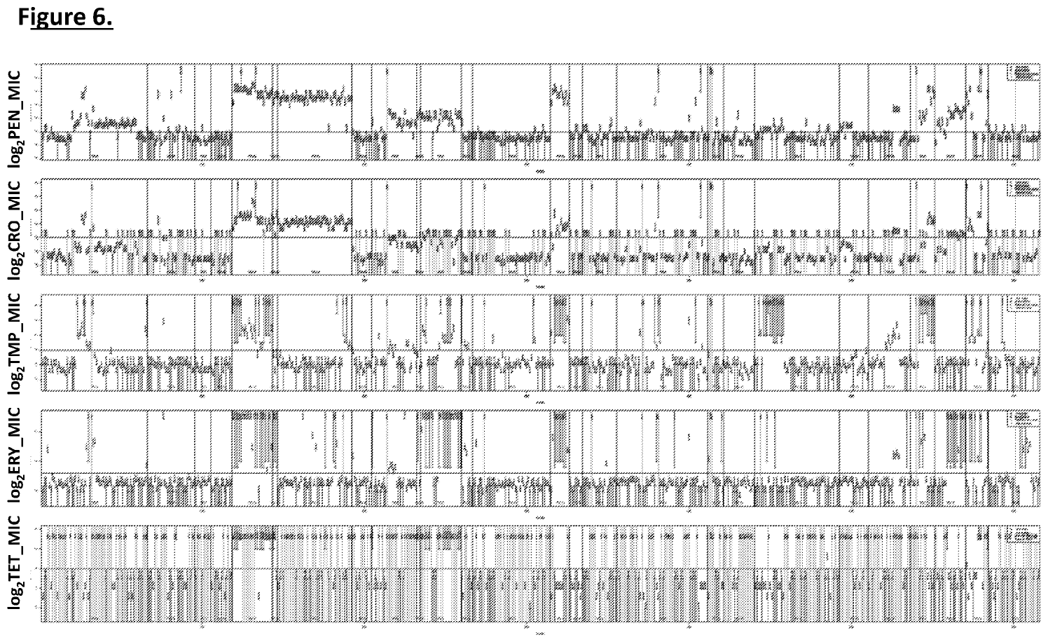

For all antibiotics, RASE associates individual isolates with a resistance category, susceptible or non-susceptible. First, MIC values are mined using regular expressions from the available textual antibiograms, i.e., strings describing an interval of possible MIC values. Second, the acquired intervals are compared to the antibiotic-specific breakpoints (FIG. 6). If a given breakpoint is above or below the interval, susceptibility or non-susceptibility is reported, respectively. However, no category can be assigned at this step if the breakpoint lies within the extracted interval, an antibiogram is entirely missing, or an antibiogram is present, but parsing failed. Third, missing categories are inferred using ancestral state reconstruction on the associated phylogenetic tree while maximizing parsimony (i.e., minimizing the number of nodes switching its resistance category; FIG. 7).

When the solution for a node is not unique, non-susceptibility is assigned.

The pneumococcal RASE database was constructed with the standard EUCAST breakpoints¹⁶ ([g/ml]): benzylpenicillin (PEN): 0.06, ceftriaxone (CRO): 0.25, trimethoprim-sulfamethoxazole (SXT): 1.00, erythromycin (ERY): 0.25, and tetracycline (TET): 1.00. The gonococcal RASE database was constructed with the CDC GISP breakpoints ([g/ml]): azithromycin (AZM): 2.0, cefixime (CFM): 0.25, ciprofloxacin (CIP): 1.0, and ceftriaxone (CRO): 0.125. While the Inventors have used the above values in the present work, others may be readily defined and the database rapidly updated. This is especially useful in the case where breakpoints may vary depending on the site of infection (as is the case with pneumococcal meningitis and otitis media, where lower MICs are considered to be resistant).

### Example 11

### Neighbor Typing

All genomes in the database are associated with similarity weights that are set to zero at the start of the run. Each time a new read is read from the stream, k-mer-based matching is applied to identify the reference genomes with the maximum number of shared k-mers (see below).

These genomes are read's nearest neighbors (NN) in the database according to the 1/(number of shared k-mers) pseudo distance.

The weight of the nearest neighbors are then increased according to the ‘information content’ of the read, calculated as the number of matched k-mers divided by the number of nearest neighbors. Reads that do not match (i.e., 0 matching k-mers in the database) are not used in subsequent analysis to predict resistance. The obtained weights are used as a basis for the subsequent prediction.

### Example 12

### K-Mer-Based Matching

Reads were matched against RASE databases using the ProPhyle classifier (commit b3881ec) and its ProPhex component. ProPhyle index stores k-mers of all genomes' assemblies in a highly compressed form, reducing the required memory footprint. In the database construction phase, the genomes' k-mers are first propagated along the phylogenetic tree and then greedily assembled to contigs. The obtained contigs are then placed into a single text file, for which a BWT-index is constructed. The obtained index can be searched for any k-mer, retrieving a list of nodes whose descending leaves correspond to genomes containing that k-mer.

In course of sequencing, each read is decomposed into overlapping k-mers, which are then localized on the tree; this is done by ProPhex using BWT-search using a rolling window with the RASE k-mer index. The obtained matches are propagated from internal nodes to the level of leaves such that read's k-mer the reference genomes in which it occurs are identified.

### Example 13

### Similarity Weights

All genomes in the database are associated with similarity weights that are set to zero at the start of the run. Each time a new read is read from the stream, it's nearest neighbors (NN) in the database are identified. This is done by k-mer-based read pseudo alignment to the RASE database using ProPhyle. The weight of the retrieved NNs is increased according to the ‘information content’ of the read, calculated as the number of matched k-mers divided by the number of NNs. Reads that do not match (i.e., 0 matching k-mers in the database) are not used in subsequent analysis to predict resistance. The obtained weights are used as a basis for the subsequent prediction.

### Example 14

### Predicting Lineage

A lineage is predicted as the lineage of the best matching reference genome. The quality of prediction is further quantified using a lineage score (LNS), which is calculated as LNS=2f/(f+t)−1, where f and t denote the scores of the best match in the first (‘predicted’) and the best match in the second (‘alternative’) lineage, respectively. The values of LNS can range from 0.0 to 1.0 with the following special cases: LNS=1.0 means that all reads were perfectly matching the predicted lineage and LNS=0.0 means that the predicted and alternative lineages were matched equally well.

RASE uses LNS to evaluate whether a sample is truly matching the database and predicting resistance for the database species makes sense. If LNS is higher than a specified threshold (0.6 in default settings), the call is considered successful. If the score is lower than this, the sample cannot be securely assigned to a lineage, and this counts as a failure. Note that custom RASE databases may require a re-calibration of the threshold.

### Example 15

### Predicting Resistance

Resistance is predicted for individual antibiotics independently, using weights of genomes within the predicted lineage and only under the condition that lineage could be detected. Resistance is predicted as the resistance of best matching reference. The confidence of the prediction is evaluated using susceptibility scores that combine the resistance characteristics of the strains in lineage being the most similar to the sample. A susceptibility score is calculated as SSC=s/(s+r), where s and r denote the weight of the best susceptible and non-susceptible strain within the predicted lineage, respectively. The values of SSC can range from 0.0 to 1.0 with the following special cases: SSC=0.0 and SSC=1.0 means that all reads match only resistance or susceptible isolates in the lineage respectively; SSC=0.5 means that the best-matching resistant and susceptible isolates within the lineage are matched equally well.

RASE uses SSC for providing the prediction as well as for evaluating the prediction's confidence. If SUS is greater than 0.5, susceptibility to the antibiotic is reported, non-susceptibility otherwise. When SSC is within the [0.4, 0.6] range, it is considered a low-confidence call. This typically happens when two genomes with different resistance categories have similar weights, which is usually the case when resistance or susceptibility emerged recently in the evolutionary history.

### Example 16

### Measuring Time

To determine how RASE works with nanopore data generated in real time, the timestamps of individual reads extracted using regular expressions from the read names. These are then used for sorting the base-called nanopore reads by time. When the RASE pipeline was applied, the timestamps were used for expressing the predictions as a function of time. The times of ProPhyle assignments were also compared to the original timestamps to ensure that the prediction pipeline was not slower than sequencing.

When timestamps of sequencing reads were not available (the gonorrhoeae WHO and clinical samples), RASE estimated the progress in time from the number of processed base pairs. This was done by dividing the cumulative bps count by the typical nanopore flow, which the Inventors had previously estimated from SP01 as 1.43 Mbps per second. However, such an estimated progress is indicative only, as it does not follow the true order of reads in course of sequencing. As the nanopore signal quality decreases over time, the randomized read order provides worse results than true real-time sequencing.

### Example 17

### Optimizing k-Mer Length

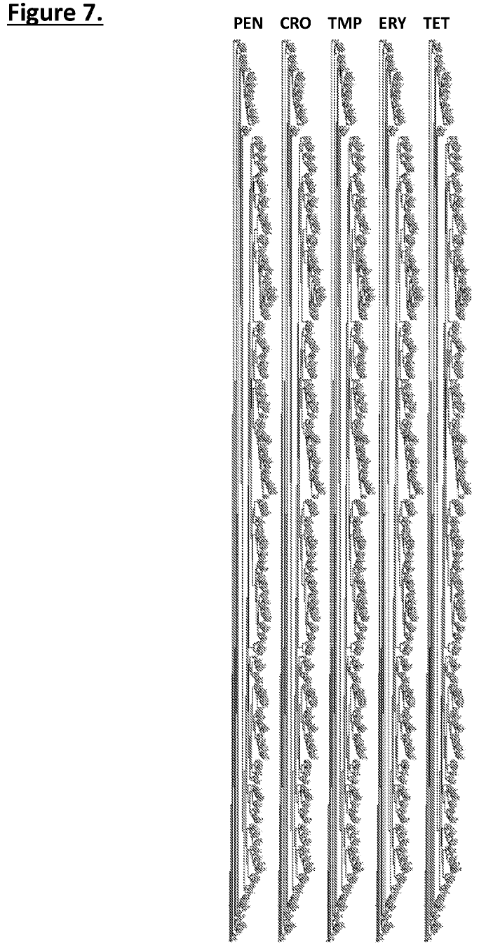

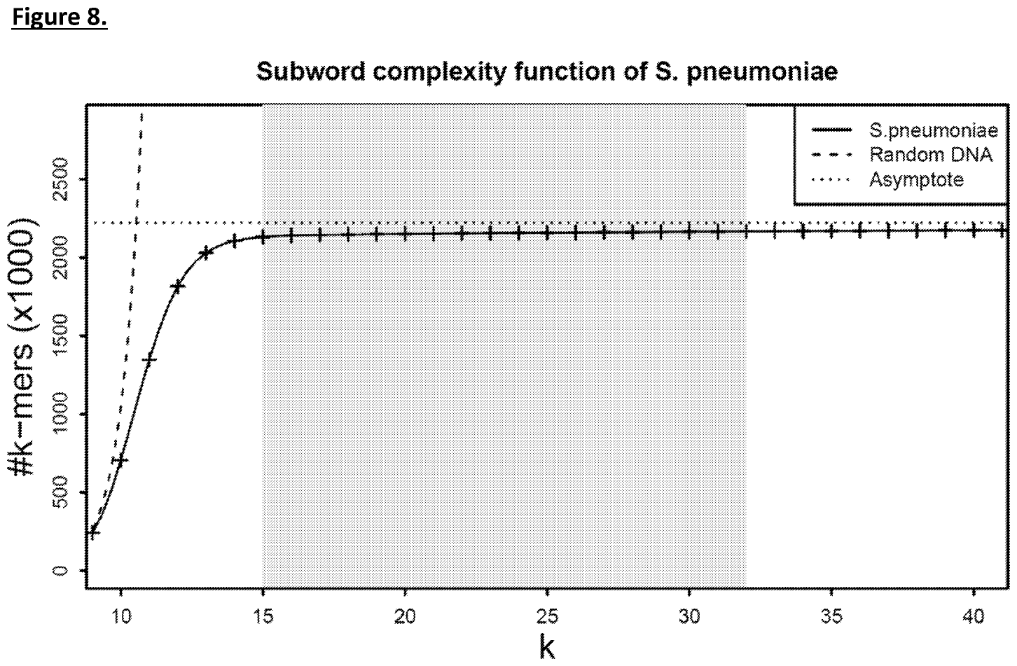

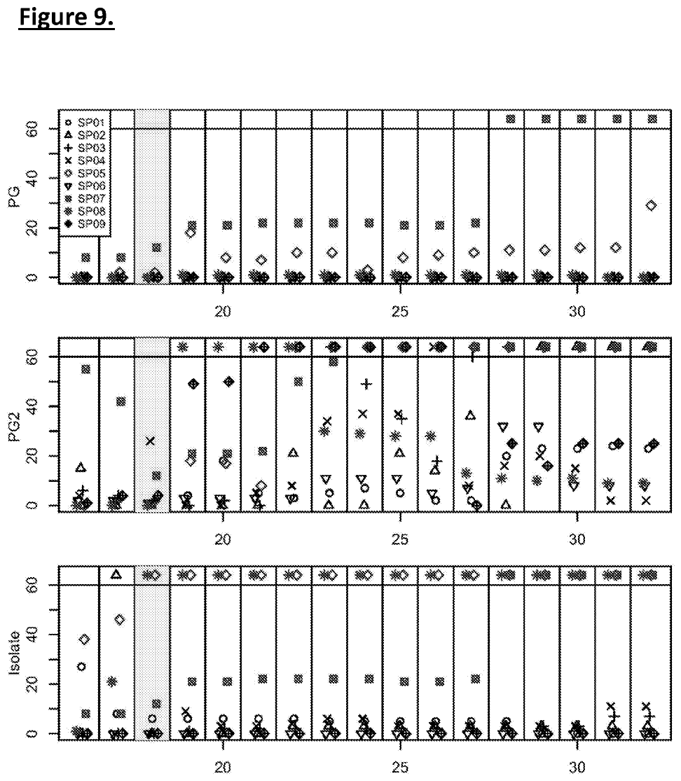

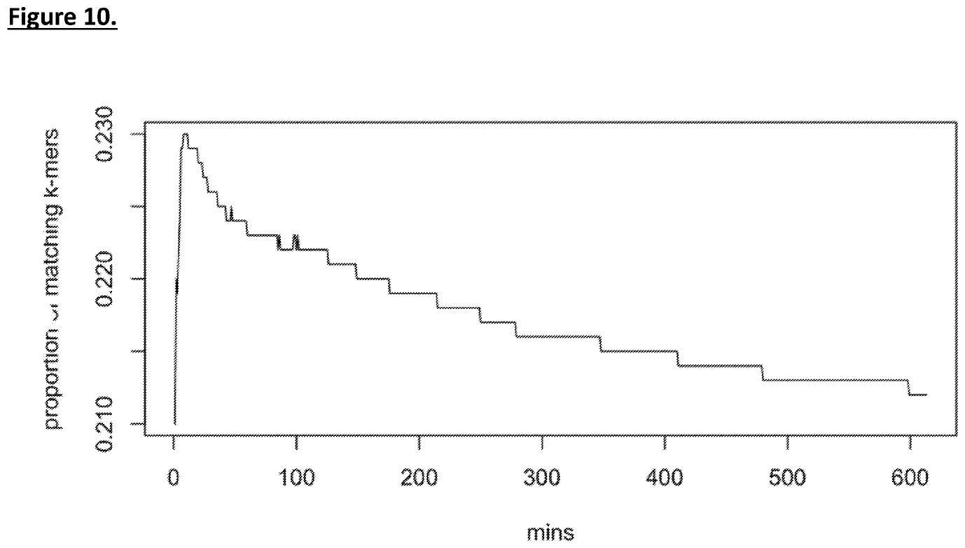

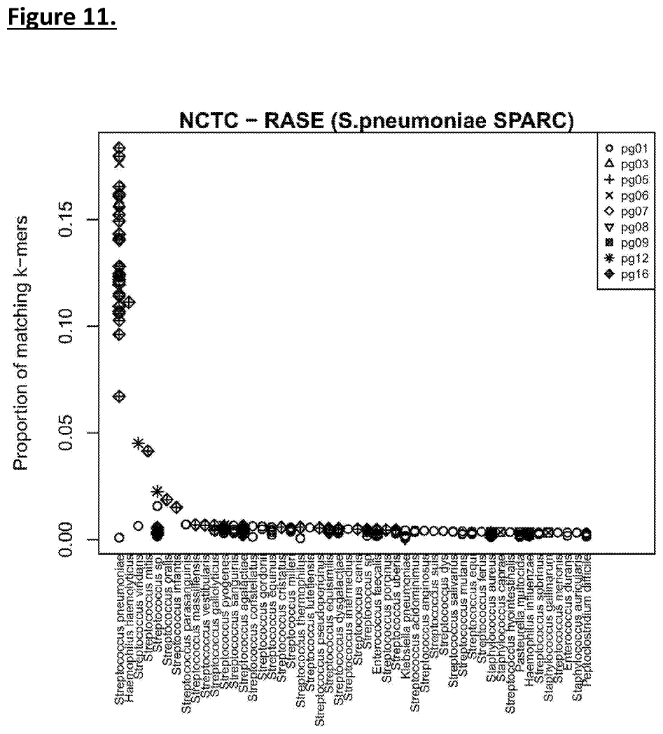

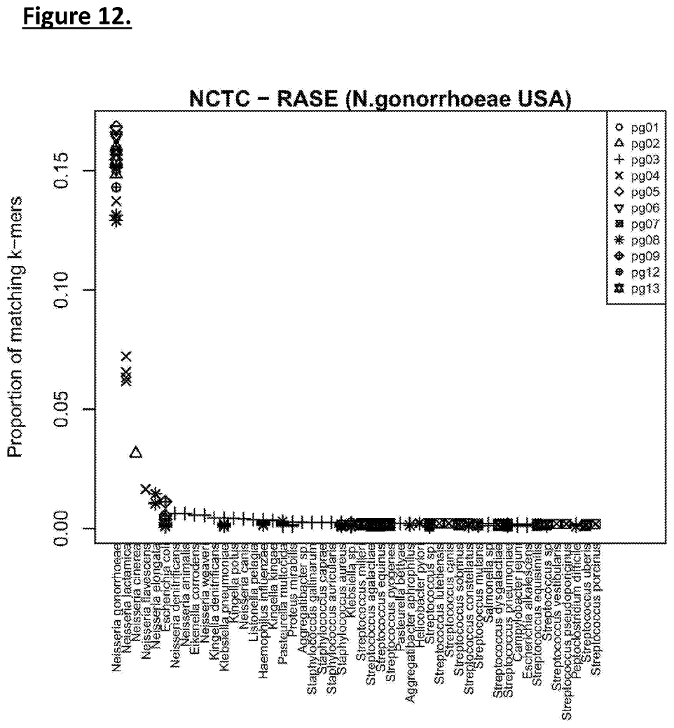

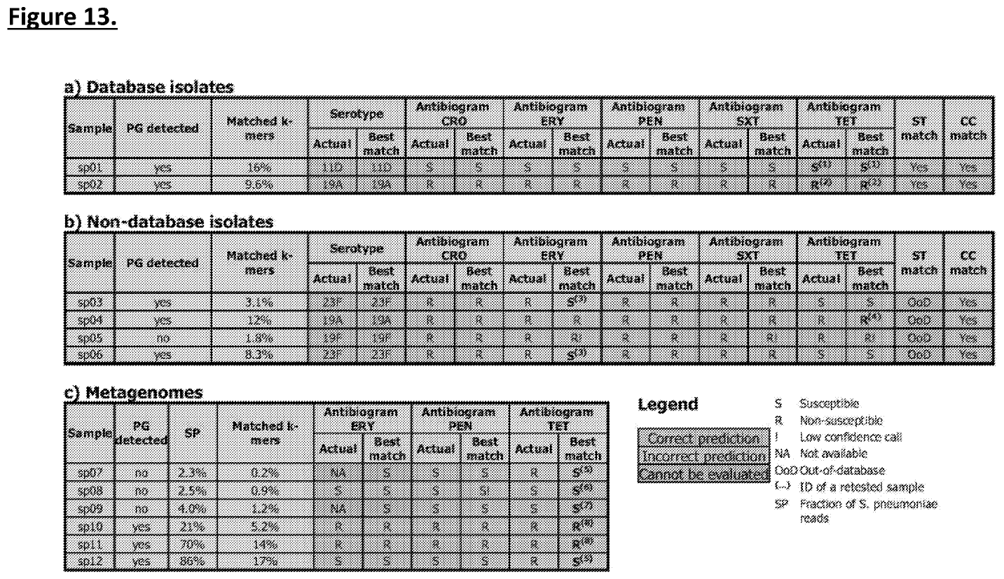

The k-mer length is the main parameter of the classification. First, the subword complexity function of pneumococcus was calculated using JellyFish (version 2.2.10) (FIG. 8). Then, based on the characteristics of the function and technical limitations of ProPhyle, the possible range of k was determined. For these k-mer lengths, RASE indexes were constructed and their performance evaluated using the RASE prediction pipeline and selected experiments. While RASE showed robustness to k-mer length in terms of final predictions, prediction delays differed (FIG. 9). Based on the obtained timing data, the Inventors set k to 18.

### Example 18

### Lower Time Bounds on Resistance Gene Detection

A complete genome assembly of the multidrug resistant SP02 isolate was computed from the nanopore reads using the CANU (version 1.5, with default parameters). Prior to the assembly step, reads were filtered using SAMsift based on the matching quality with the RASE database: only reads at least 1000 bp long with at least 10% 18-mers shared with some of the reference draft assemblies were used. The obtained assembly was further corrected by Pilon (version 1.2, default parameters) using Illumina reads from the same isolate (taxid ‘QJAP’ in the SPARC dataset) mapped to the nanopore assembly using BWA-MEM (version 0.7.17, with the default parameters) and sorted using SAMtools.

The obtained assembly was searched for resistance-causing genes using the online CARD tool (as of 2018/08/01). All of the original nanopore reads were then mapped using Minimap2 (version 2.11, with ‘-x map-ont’) to the corrected assembly and resistance genes in the reads identified using BEDtools-intersect (version 2.27.1, with ‘-F 95’). Timestamps of the resistance-informative reads were extracted and associated with the genes. Only reads longer than 2 kbp were used in the analysis.

### Example 19

### Evaluation of the N. gonorrhoeae WHO Samples

To evaluate the predictions of the WHO samples, the Inventors inferred a phylogenetic tree from a data set comprising the GISP isolates and the WHO isolates. Read data were downloaded for the GISP isolates (accession numbers: PRJEB2999 and PRJEB7904) and for the WHO isolates F-P (accession number: PRJEB4024). For the WHO isolates U-Z, read data were simulated from the finished de novo assemblies (accession number: PRJEB14020) using Art (version 2.5.1).

Reads were mapped to the NCCP11945 reference genome (GenBank accession: CP001050.1) using BWA-MEM (version 0.7.17) (ref) and deduplicated using Picard (version 2.8.0) (refs). Pilon (version 1.16, with ‘--mindepth 10 --minmq 20’) (ref) was used to call variants and further filtered to include only “pass” sites and sites where the alternate allele was supported with AF >0.9 (ref). Gubbins (version 2.3.4) with RAxML (version 8.2.10) were run on the aligned pseudogenomes to generate the final recombination-corrected phylogeny.

The correctness of the RASE assignments was verified using the obtained tree. For every WHO isolate, the obtained RASE prediction was compared to the closest GISP isolate on the tree.

### Example 20

### Library Preparation

For experiments SP01-SP06, cultures were grown in Todd-Hewitt medium with 0.5% yeast extract (THY; Becton Dickinson and Company, Sparks, Md.) at 37° C. in 5% CO2 for 24 hrs. High molecular weight (>1 ug) genomic DNA was extracted and purified from cultures using DNeasy Blood and Tissue kit (QIAGEN, Valencia Calif.). DNA concentration was measured using Qubit fluorometer (Invitrogen, Grand Island N.Y.). Library preparation was performed using the Oxford Nanopore Technologies 1D ligation sequencing kit SQK LSK108.

For experiments SP07-SP12, library preparation was performed using the ONT Rapid Low-Input Barcoding kit SQK-RLB001, with saponin-based host DNA depletion used for reducing the proportion of human reads.

For sequenced gonococcal strains GCGS0092, GCGS0938, and GCGS1095, cultures were grown on Chocolate-Agar media i.e., Difco GC base media containing 1% IsoVitaleX (Becton Dickinson Co., Franklin Lakes, N.J.) and 1% Remel Hemoglobin (Thermo Fisher Scientific, Carlsbad, Calif.) at 37° C. in 5% CO2 for 20 hrs. Genomic DNA was extracted and purified from cultures using the PureLink Genomic DNA MiniKit (Thermo Fisher Scientific, Carlsbad, Calif.). DNA concentration was measured using the Qubit fluorometer (Invitrogen, Grand Island, N.Y.). Library preparation was performed using the Oxford Nanopore Technologies 1D ligation sequencing kit SQK-LSK109.

### Example 21

### MinION Sequencing

Sequencing was performed on the MinION MK1 device using R9.4/FLO-MIN106 flowcells, according to the manufacturer's instructions. For experiments SP01-SP06, base-calling was performed using ONT Metrichor (versions 1.6.11 (SP01), 1.7.3 (SP02), 1.7.14 (SP03-SP06)) simultaneously with sequencing and all reads passing Metrichor quality check were used in the further analysis. For experiments SP07-SP12, ONT MinKNOW software (versions 1.4-1.13.1) was used to collect raw sequencing data and ONT Albacore (versions 1.2.2-2.1.10) was used for local base-calling of the raw data after sequencing runs were completed. For experiments GCGS0092, GCGS0938, and GCGS1095, ONT MinKNOW software was used to collect raw sequencing data and ONT Albacore (versions 2.3.4) was used for local base-calling.

### Example 22

### Testing Resistance Phenotype

Additional retesting of SPARC isolates was done using microdilution. Organism suspensions were prepared from overnight growth on blood agar plates to the density of a 0.5 McFarland standard. This organism suspension was then diluted to provide a final inoculum of 10⁵ to 10⁶ CFU/ml. Microdilution trays were prepared according to the NCCLS methodology with cation-adjusted Mueller-Hinton broth (Sigma-Aldrich) supplemented with 5% lysed horse blood (Hemostat Laboratories). Penicillin (TRC Canada) and chloramphenicol (USB) concentrations ranged from 0.016 to 16 μg/ml. Erythromycin (Enzo Life Sciences), tetracycline (Sigma-Aldrich), and trimethoprim-sulfamethoxazole (MP Biomedicals) concentrations ranged from 0.0625 to 64 μg/ml. Ceftriaxone (Sigma-Aldrich) concentrations ranged from 0.007 to 8 μg/ml. The microdilution trays were incubated in ambient air at 35° C. for 24 h. The MICs were then visually read and breakpoints applied. A list of individual microdilution measurements and the obtained resistance categories is provided.

Resistance of streptococcus in the metagenomic samples (SP07-SP12) was determined by agar diffusion using the EUCAST methodology and breakpoints. First, the inoculated agar plates were incubated at 37° C. overnight and then examined for growth with the potential for re-incubation up to 48 hours. Then, the samples were screened to oxacillin: if the zone diameter r was >20 mm, the isolate was considered sensitive to benzylpenicillin, otherwise a full MIC measurement to benzylpenicillin was done. Finally, the isolate was screened for resistance to tetracycline (r>25 mm for sensitive, r<22 mm for resistant) and erythromycin (r>22 mm for sensitive, r<19 mm for resistant); when the isolate showed intermediate resistance, a full MIC measurement was done.

### Example 23

### Data, Implementation and Availability

RASE was developed using Python, GNU Make, GNU Parallel, Snakemake, and the ETE 3 and PySam libraries, and was based on ProPhyle v0.3.1.3. Bioconda was used to ensure reproducibility of the software environments. All code and the generated database are available under the MIT license from http://github.com/c2-d2/rase. Sequencing data for all experiments can be downloaded from http://doi.org/10.5281/zenodo.1405173; for the metagenomic experiments, only the filtered datasets (i.e., after removing the remaining human reads in silico) were made publicly available.

### Example 24

### Resistance is Strongly Clonal in S. pneumoniae and N. gonorrhoeae

The Inventors first studied whether antibiotic resistance is associated with particular lineages of the pathogens S. pneumoniae and N. gonorrhoeae. Lineages of S. pneumoniae and N. gonorrhoeae are predictive for resistance with Area under the Receiver Operation Characteristic Curve (AUROC) ranging from 0.90 to 0.97. In case of the S. pneumoniae, the AUROCs for benzylpenicillin, ceftriaxone, trimethoprim-sulfamethoxazole, erythromycin, and tetracycline were 0.90, 0.95, 0.90, 0.90, and 0.97 respectively, consistent with previous observations. In N. gonorrhoeae, the AUROCs for azithromycin, ciprofloxacin, ceftriaxone, and cefixime were 0.80, 0.98, 0.93, and 0.97, respectively. These strong associations suggest that resistance of a clinical specimen could be predicted from the position of bacteria in the phylogeny, which can be determined from sequencing data.

### Example 25

### Rapid Identification of Nearest Known Relative from Sequencing Reads

The Inventors developed an approach that the Inventors term ‘neighbor typing’ to predict phenotype from sequencing data. Neighbor typing is a two-step algorithm, which first compares a provided sample to a database of reference genomes with a known phylogeny and phenotype, and then predicts the likely phenotype of the sample under test based on the best hits and their matching quality. The Inventors apply this here to the detection of drug resistance.

To implement neighbor typing the Inventors developed a software called RASE (Resistance-Associated Sequence Elements) (FIG. 1). RASE takes a stream of nanopore reads and compares them to references using k-mer-based matching using a modified version of ProPhyle. ProPhyle uses Burrows-Wheeler Transform and FM-index to implement a fast and memory-efficient exact colored de-Bruijn graph data structure, which subsequently allows us to rapidly and accurately estimate sample-to-reference sequence similarity. Based on the obtained read k-mer matches, RASE identifies the read's nearest neighbors in the database and increases their similarity weights. These are cumulative scores capturing sample-to-reference similarity; they are set to zero at the beginning and are increased on-the-fly as sequencing proceeds according to each read's ‘information content’. Generally speaking, longer reads, such as those covering multiple accessory genes, tend to be specific and have high scores; whereas short reads or reads from the core genome tend to be non-specific and have low scores, being found in many genomes.

Predictions are done in two steps. First, RASE predicts a lineage as the lineage of the best matching reference genome and estimates the confidence of lineage assignment by comparing the two best matching lineages to compute a ‘lineage score’. Second, RASE goes further by identifying the genomes that are the closest relatives of the specimen, and then predicts resistance from the nearest resistant and susceptible neighbor within the lineage.

Comparison of these provides a ‘susceptibility score’, which quantifies the risk of resistance. When these are too similar, the call's confidence is considered low—this happens especially when resistance emerged recently in evolutionary history. The ability to pinpoint the closest relatives in the database offers further resolution, even in the case where the resistance phenotype varies within a lineage.

Results of RASE are reported in real time as the best matching genome in the database, together with the predicted lineage and its score, susceptibility scores to the antibiotics being tested, and a proportion of matching k-mers for quality control. As the run progresses, the scores fluctuate and eventually stabilize (examples shown in FIG. 2).

### Example 26

### RASE Databases for Hundreds of S. pneumoniae and N. gonorrhoeae Isolates

The Inventors constructed RASE databases for S. pneumoniae and N. gonorrhoeae. First, the Inventors used 616 pneumococcal genomes from a carriage study in Massachusetts children. Second, the Inventors used 1102 clinical gonococcal isolates collected from 2000 to 2013 by the Centers for Disease Control and Prevention's Gonococcal Isolate Surveillance Project. In both cases, the datasets comprised draft genome assemblies from Illumina HiSeq reads, resistance data, and genome clusters computed using Bayesian Analysis of Population Structure (BAPS).

The Inventors assigned each pneumococcal and gonococcal isolate to an antibiotic-specific resistance category using the EUCAST breakpoints and CDC GISP breakpoints, respectively. Since MIC data were not always available, the Inventors estimated the likely resistance phenotype of unannotated isolates using ancestral state reconstruction. The Inventors tested eight pneumococcal isolates for which resistance was not originally available and the measured MICs by microdilution matched the phenotypes provided by ancestral state reconstruction (shown in bold in FIG. 14).

### Example 27

### RASE Identifies Isolates within the Database in Minutes

The Inventors examined two pneumococcal and five gonococcal isolates that were used to build the RASE database (FIG. 14a) to test whether the Inventors can correctly assign lineage under ideal circumstances. For SP01 the correct lineage and matching isolate were identified within 1 minute and 7 minutes respectively (FIG. 2). The SP02 isolate was predicted even faster, with both lineage and best match correctly detected and stabilized within 1 minute. Therefore, neighbor typing can be accurate and fast even using sequence data with a high per-base error rate.

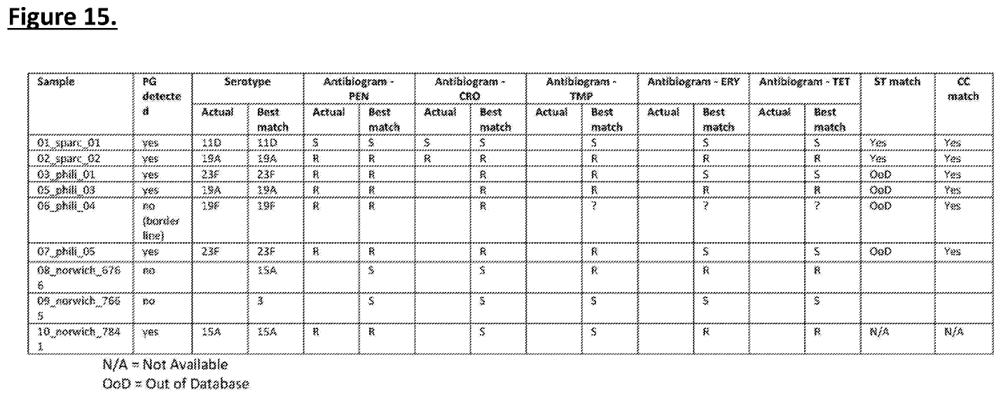

The Inventors performed a similar evaluation with five gonococcal isolates (FIG. 15a). First, the Inventors tested a fully sensitive isolate (GC02); here RASE identified the correct isolate and antibiogram within 3 minutes of sequencing. The Inventors then sequenced an isolate with a novel and uncommon mechanism of cephalosporin resistance that has emerged recently (GC03). Under such circumstances, the resistant isolate and its susceptible neighbors tend to be genetically very similar, which could confound the Inventors' analysis. However, RASE was still able to identify the correct antibiogram in 9 minutes, with the delay being due difficulty distinguishing between the close relatives, reflected also by the susceptibility score in the low-confidence range. This was repeated in further experiments with the same isolate which consistently reported low confidence in resistance phenotype which would draw operators' attention and indicate further testing was necessary. For the multi-drug resistant isolate (GC05) RASE predictions stabilized within 2 minutes but incorrectly susceptibility to ceftriaxone. A subsequent analysis revealed that the ceftriaxone MIC of the sample was equal to the CDC GISP breakpoint (0.125) whereas the best match in the database had an MIC of 0.062; within a single doubling dilution (need citation for this?). The Inventors found that RASE performed well even with extremely poor data and low-quality reads.

### Example 28

### RASE Identifies the Closest Relative of Novel Isolates

The Inventors examined four additional pneumococcal isolates (FIG. 14b) for which the serotype and limited antibiogram and lineage data were known. The Inventors compared three characteristics of the sample to assess the Inventors' performance: the serotype, the MLST sequence type (ST) and the antibiograms (benzylpenicillin, ceftriaxone, trimethoprim-sulfamethoxazole, erythromycin, and tetracycline resistance according to EUCAST breakpoints).

In all cases, the closest relative was identified within 5 minutes, even if the correct ST was absent from the RASE database, indicating the strength of the neighbor typing method in rapidly detecting similarity. The two 23F samples (SP03 and 5P06) were correctly called as being closely related to the Tennessee 23F-4 clone identified by PMEN, a clone strongly associated with macrolide resistance. Consistent with this, the two samples were indeed resistant to erythromycin. However, the Tennessee 23F-4 clone was absent from the Massachusetts sample, with the best match being a comparatively distantly related isolate that was penicillin resistant, but erythromycin susceptible, hence correctly identifying only part of the antibiogram. This illustrates the importance of a relevant sample from which to construct the RASE database. In the case of 5P05, the lineage score was borderline, reflecting divergence of the sample under test from the database, even though in this case the susceptibility scores were accurate for the antibiotics tested.

The Inventors performed a similar evaluation with 14 clinical gonococcal isolates not present in the RASE database. To assess RASE capabilities to predict resistance in a hospital setting, the Inventors applied RASE to 14 clinical gonococcal isolates from the RaDAR-Go project (Switzerland, 2015-2016) that were previously sequenced using nanopore and for which full antibiograms are available (FIG. 15b). In case of the incorrect susceptibility call to azithromycin, RASE reported a low-confidence call. These results show that gonococcal RASE databases built in the US may be applicable in Europe.

### Example 29

### Phenotyping is Still Informative but Lower Quality on Divergent Lineages

As noted above an important precondition of neighbor typing is a comprehensive and relevant reference database. To evaluate how RASE performs in a borderline setting with lineages that are not sufficiently represented in the GISP database, the Inventors used the gonococcal WHO 2016 reference strain collection. This includes a global collection of 14 diverse isolates from Europe, Asia, North America, and Australia, collected over two decades and exhibiting phenotypes ranging from pan-susceptibility to multi-drug resistance. The WHO strains are available from the National Collection of Type Cultures, and were previously sequenced using nanopore and genetically and phenotypically characterized. Surprisingly, RASE correctly identified all STs represented in the database and in 7 cases it provided fully correct antibiograms. In 6/7 cases where the complete resistance profile was not recovered, the closest neighbors were identified correctly but were genetically divergent from the query isolates (Supplementary Note 3). In one case, the errors were due to a misidentification of the correct part of the phylogeny by ProPhyle. Therefore, most prediction errors were due to the fact that sufficiently close relatives of these isolates were not present in the Inventors' database, which could be fixed with a more comprehensive database.

### Example 30

### RASE can Identify Resistance in Pneumococcus from Sputum Metagenomic Samples

Because bacterial culture introduces significant delays, direct metagenomic sequencing of clinical samples would be preferable for point-of-care use. The Inventors therefore analyzed metagenomic nanopore data from sputum samples obtained from patients suffering from lower respiratory tract infections, selecting 6 samples from the study that were already known to contain S. pneumoniae (FIG. 14c).

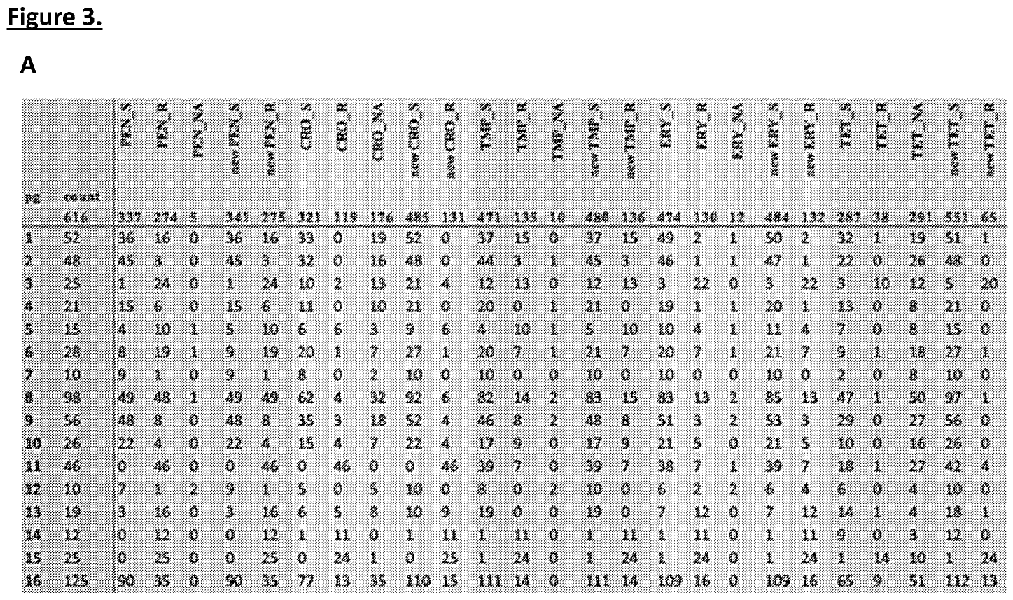

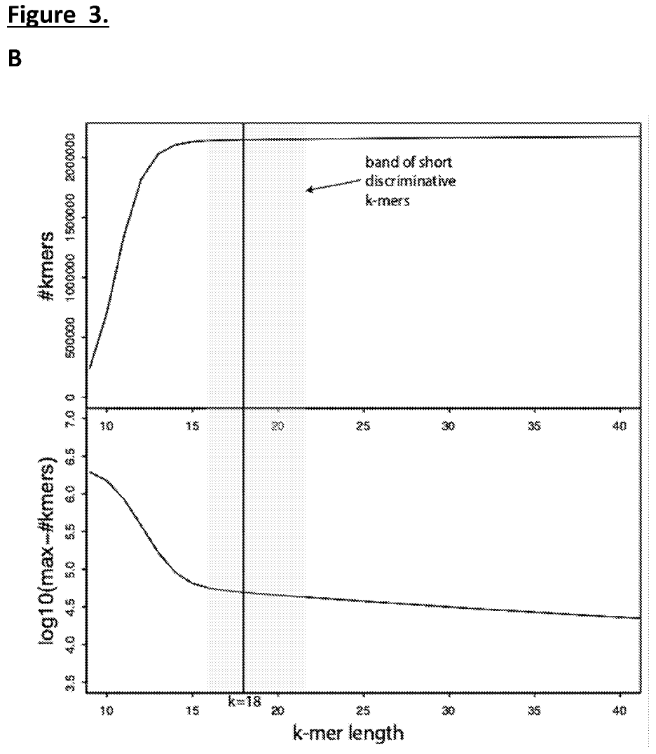

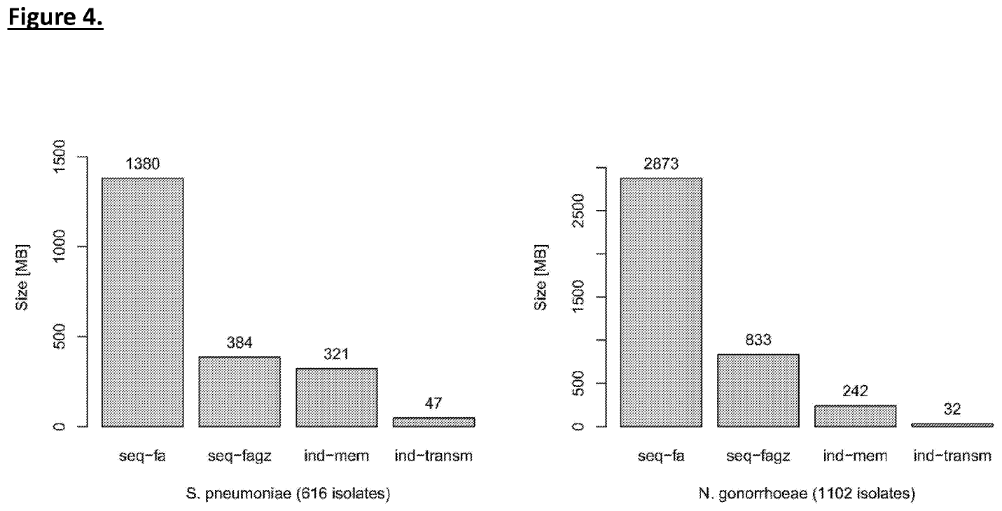

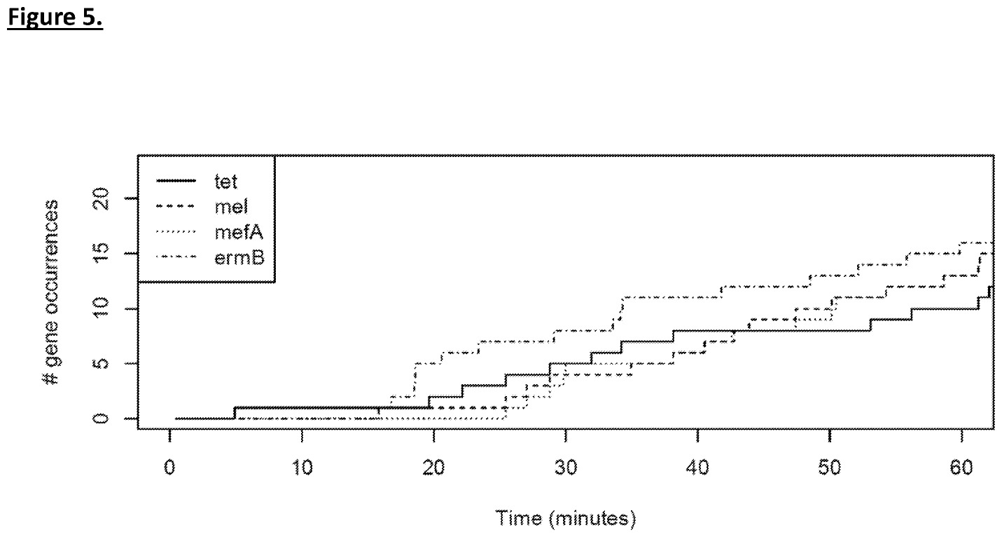

One sample (SP10) contained DNA from multiple bacterial species (FIG. 3). However, within 5 minutes sequence was identified belonging to the Swedish 15A-25 clone (ST63) which is also known to be associated with resistance phenotypes including macrolides and tetracyclines.

This sample was confirmed to be resistant to erythromycin, as well as clindamycin, tetracycline and oxacillin according to EUCAST breakpoints. The original report of the Swedish 15A-25 clone did not report resistance to penicillin antibiotics, which has subsequently emerged in this lineage. However, the Inventors' database correctly identified the risk of penicillin resistance in this sample. The metagenomes SP11 and SP12 contain an estimated >20% reads that matched to S. pneumoniae, and their serotypes were identified to be 15A and 3, respectively. The susceptibility scores of the best matches were fully consistent with the susceptibility profiles found in the samples, with the exception of tetracycline resistance in SP12 due to an incomplete database. The last remaining samples, SP07-SP09, contained less than 5% unambiguously pneumococcal reads, and as a result the lineage was not securely identified in these. Nevertheless, all predicted phenotypes were concordant with phenotypic tests, with the exception of SP07 which matches the same isolate as SP12 (discussed above).

### Example 31

### Additional Information

Further analysis of the reads from SP12 using Krocus44 suggested that the pneumococcal DNA present was from the ST180 clonal complex, and matched specifically either to the sequence type ST180 or ST3798. This is consistent with identification as serotype 3, because this clonal complex contains the great majority of isolates with this capsule type, which historically has not been associated with resistance45. However, improved sampling and study of this lineage has recently found highly divergent subclades that are associated with resistance. These lineages were previously rare, and thus were less likely to be included in the Inventors' database, but now are increasing in frequency. In this case, ST3798 is found to be in clade 1B, which is notable for exhibiting sporadic tetracycline resistance. Again, the failure to match to this is a result of the original database not containing a suitable example for comparison.

The Inventors evaluated how long it took for resistance genes to be reliably detected in nanopore reads. For SP02 the Inventors observed that at least 15 minutes were needed to detect resistance, assuming that the genes in question can be unambiguously identified in nanopore data despite the high per base error rate, and that the presence of the loci is directly linked to the resistance phenotype. If this is not the case, further delays would be expected. Thus, neighbor typing can offer a time advantage compared to methods based on identifying the presence of resistance genes even in a sample of DNA from a purified isolate as opposed to a metagenome, potentially allowing for more rapid changes to antimicrobial therapy.

The Inventors analyzed the results of the WHO gonococcal samples. First, the Inventors evaluated the RASE ability to predict MLST types. In all cases, either RASE predicted the correct sequence type (n=9), or the true sequence was not present in the reference database (n=5). The latter was the case only in the samples F through P, which belonged to the initial 2008 WHO reference panel and were collected primarily in the late 1990s, with the majority of specimens isolated from the Eastern Hemisphere47. The GISP database, comprising strains collected in the US from 2000-2013, may not be representative then of the circulating lineages in those regions during that time span, which could result in both ST and antibiogram prediction errors. The Inventors observed perfect prediction of MLSTs in the additional 2016 WHO reference strains comprising U through Z that were collected in 2007 and onwards.

The Inventors next sought to evaluate the resistance predictions. In 7 cases (F, K, N, O, P, U, W), the antibiograms were identified fully correctly; in 4 (G, V, X, Z) and 3 cases (L, M, Y) one and two mistakes were made, respectively. To explain these discrepancies, the Inventors inferred a recombination-corrected phylogenetic tree comprising the GISP database isolates as well as the WHO samples (Supplementary Newick file). With the exception of G and Y, the WHO isolates and their respective RASE-predicted best matches were the closest GISP isolates, indicative of accurate matching by RASE. While branch lengths of L, M and V on the tree reveal that the corresponding parts of the phylogeny are not well sampled in the database, the X, Y, and Z samples emerged from lineages that are well-represented, but have acquired an atypically high level of cephalosporin resistance. Whereas X and Z acquired a novel resistance-conferring mosaic penA allele48, Y acquired a novel active site mutation in the context of a pre-existing mosaic penA allele49. While both of these adaptations resulted in high-level resistance, these mutations also appear to incur fitness costs in vitro and in the gonococcal mouse mode150. In line with this, these strains have only been sporadically observed in genomic surveillance of clinical isolates. These results therefore highlight how ancestral or emerging resistant lineages may not be well-captured by RASE and emphasize the importance of continuous updating of the RASE database.

The Inventors evaluated how RASE performs in extremely unfavorable sequencing conditions; the Inventors sequenced a fully susceptible isolate from the database with the use of old reagents and obtained in consequence only 3.5 Mbps of low quality reads (only 7% of matching k-mers compared to 20% obtained in the other isolates) (GC01 in FIG. 15a). An experiment with such a low yield would normally be discarded; despite that RASE provided correct and stabilized predictions (once first long read was obtained from the sequencer at t=21 mins), with the exception of oscillating azithromycin score, which reflected that resistance to azithromycin has emerged recently.

Following the analysis of the S. pneumoniae genome and the characteristics of nanopore reads, the Inventors set the Inventors' k-mer length to 18. Such k-mers are short compared to standard methodologies51,52, but offer higher robustness to the high error rates in nanopore sequencing and bacterial within-species variation. The Inventors' constructed pneumococcal and gonococcal ProPhyle k-mer databases occupy 320 MB and 443 MB RAM (4.3× and 6.9× compression rate) and can be further compressed for transmission to 47 MB and 64 MB (29× and 45× compression rate), respectively (Supplementary FIG. 1). This demonstrates that RASE can be used on portable devices and its databases easily transmitted to the point of care over links with a limited bandwidth.

Out of all 616 pneumococcal isolates, 341 were associated with susceptibility to benzylpenicillin, 485 to ceftriaxone, 480 to trimethoprim-sulfamethoxazole, 484 to erythromycin, and 551 to tetracycline. In case of gonococcus, ancestral reconstruction was needed only for cefixime (62 records). Out of all 1102 gonococcal isolates, 232 were associated with resistance to azithromycin, 594 to ciprofloxacin, 69 to ceftriaxone, and 266 to cefixime. In the Inventors' subsequent experiments, if original MIC data were not available for the best match in the RASE database, the relevant isolate was tested to confirm resistance phenotype.

### Example 32

### Discussion

This paper presents a method the Inventors term neighbor typing to pinpoint the closest relatives of a query genome within a suitable database, and then infer the phenotypic properties of the bacteria under test on the basis of the properties of those relatives. At present, the precise lineage of a bacterial pathogen is determined late in the day, once most important clinical decisions have been made, but adding neighbor typing at an earlier stage offers a way of leveraging bacterial population structure to gain extra information to inform treatment by identifying the presence of a high-risk pathogen in a sample. The results from the metagenomic samples suggest that it is possible to apply this approach directly to clinical samples, and the application to two very different pathogens indicate that it may have wide application.

The two pathogens studied here present contrasting features; the gonococcus is Gram-negative, harbors plasmids, and has a strikingly uniform core genome, while the pneumococcus is Gram-positive, does not contain plasmids and is diverse in both its core and accessory genome. Both exhibit high rates of homologous recombination which is expected to both spread chromosomally encoded resistance elements, and to scramble the phylogenetic signal that the Inventors use to identify the lineage. Despite these differences, and the presence of recombination, the Inventors' approach performs similarly with both pathogens, with some differences that indicate opportunities and limitations for the application.

The initial identification of the precise genome which is the closest relative is consistently more secure in the pneumococcus than the gonococcus, as a result of the former having more k-mers that are specific to an individual lineage (as a result of the greater sequence diversity mentioned above). This is not the case in the gonococcus as a result of the much lower sequence diversity in this species. As a result, in some cases (GC01 or GC04) where multiple closely related genomes are present in the database the Inventors fluctuate between them, even though the Inventors correctly identify the region of the phylogeny. If these genomes vary in their susceptibility profile, this is properly reflected in an uncertain susceptibility score indicating that caution and further investigation are merited.

For the pneumococcus the principal limitation in identifying high risk strains with neighbor typing is whether the strains are present in the database. Similar to methods that apply machine learning to a database to identify the correlates of a phenotype of interest, it is necessary that they be present in order to be learned. While the Inventors have made use of a relatively small sample from a limited geographic area to demonstrate proof of principle, in practice there are multiple examples of large genome databases generated by public health agencies, which could be combined with metadata on resistance for neighbor typing. Such databases could if necessary be supplemented with local sampling. The relevant question for the Inventors' approach therefore becomes whether the database contains a sufficiently high proportion of strains that will be encountered in disease. Further work is required to determine the optimal structure and contents of databases for each application, but the Inventors emphasize the range of pathogens which appear to show promise for this approach. However, neighbor typing may be less suitable with the current technologies in the case where there is little genomic variation (e.g., Mycobacterium tuberculosis) or not suitable at all when resistance emerges rapidly on independent and diverse genomic backgrounds (e.g., Pseudomonas aeruginosa).

Another limitation is the time required for sample preparation, which currently includes human DNA depletion, DNA isolation and library preparation, taking a total of 4 hours. This is a rapidly evolving area of technology: ONT Voltrax technology already offers automated library preparation, and the recently developed Rapid Sequencing Kit allows library preparation in 10 minutes. Further advances in this space, in particular for the preparation of metagenomic samples, will be required to bring the method closer to the bedside.

Effective methods for detecting resistance, or susceptibility, from gene sequences do not need to perform GWAS in reverse—using neighbor typing, there is no requirement to detect the variation that causes the phenotype, only that it be sufficiently strongly associated with the phenotype to make reliable predictions. A key advantage of this approach is that it requires very little information, thus is not limited by high error rates or low coverage; it is not attempting to define the exact genome sequence of the sample being tested, but merely which lineage it comes from.

Neighbor typing can also be used to detect other phenotypes that are sufficiently tightly linked to a phylogeny, for instance virulence. Further applications may include rapid outbreak investigations, as the closely related isolates involved in the outbreak will all be predicted to match to the same strain in the RASE database. The approach also lends itself to enhanced surveillance, including field work situations; the recent Ebola outbreak in West Africa, for example, saw MinION devices used in remote locations without advanced healthcare facilities. Finally, this approach is not at present intended to supplant empiric therapies and prescriptions should be made as early as possible. However, the Inventors may be able to institute effective therapy at the second dose when the initial therapy is inadequate, long before it would become clinically apparent the patient is not responding. The combination of high-quality RASE databases with neighbor typing hence offers an alternative model for diagnostics and surveillance, with wide applications for the management of infectious disease.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to strain identification, including sequencing and isolation techniques related to genetic material of strains, including pathogenic or antibiotic resistant strains. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of classifying properties of one or more biological strains in a sample, comprising:
providing a biological sample comprising one or more biological strains;
sequencing DNA in the biological sample;
comparing one or more phylogroups to DNA sequences of at least two loci in the biological sample, wherein the phylogroup is associated with one or more properties; and
classifying the one or more biological strains into the one or more phylogroups, thereby classifying properties of biological strains in the sample.

2. The method of claim 1, wherein the biological sample is metagenomic.

3. The method of claim 1, wherein sequencing has up to 20% error.

4. The method of claim 1, wherein sequencing provides data in a real-time stream.

5. The method of claim 1, wherein the one or more phylogroups comprises an index of nucleotide sequences, each of at least 15 nucleotides in length.

6. (canceled)

7. The method of claim 1, wherein classifying the one or more biological strains into the one or more phylogroups comprises weighted scoring of the sequences of the at least two loci.

8. (canceled)

9. (canceled)

10. The method of claim 1, wherein the one or more properties comprise one or more of: antibiotic resistance, pathogenicity, and serotype.

11. The method of claim 1, wherein the one or more biological strains are bacteria, viruses, or fungi.

12. (canceled)

13. (canceled)

14. (canceled)

15. A method of therapeutic selection, comprising:
providing a biological sample isolated from a subject, wherein the biological sample comprises one or more biological strains;
sequencing DNA in the biological sample;
comparing one or more phylogroups to DNA sequences of at least two loci in the biological sample, wherein the phylogroup is associated with one or more properties;
classifying the one or more biological strains into the one or more phylogroups, thereby classifying properties of biological strains in the sample;
selecting a therapeutic agent based on the properties of biological strains in the subject; and
administering the therapeutic agent to the subject.

16. The method of claim 15, wherein the biological sample is metagenomic.

17. The method of claim 15, wherein the one or more phylogroups comprises an index of nucleotide sequences, each of at least 15 nucleotides in length, wherein classifying the one or more biological strains into the one or more phylogroups comprises weighted scoring of the sequences of the at least two loci, and further wherein weighted scoring comprises higher weighting for longer sequences and/or sequences covering multiple accessory genes.

18. The method of claim 15, wherein the one or more biological strains are bacteria and the properties comprise antibiotic resistance.

19. The method of claim 18 wherein selecting a therapeutic agent comprises choosing an antibiotic, wherein the one or more biological strains are susceptible to the antibiotic.

20. (canceled)

21. A method of rapid screening of a biological sample, comprising:
providing a biological sample isolated from a subject, wherein the biological sample comprises one or more biological strains;
sequencing DNA in the biological sample;
comparing one or more phylogroups to DNA sequences of the at least two loci in the biological sample, wherein the phylogroup is associated with one or more properties; and
classifying the one or more biological strains into the one or more phylogroups, thereby classifying properties of biological strains in the sample, wherein sequencing has up to 20% error and provides data in a real-time stream, wherein the one or more phylogroups comprises an index of nucleotide sequences, each of at least 15 nucleotides in length, wherein classifying the one or more biological strains into the one or more phylogroups comprises weighted scoring of the sequences of the at least two loci with higher weighting for longer sequences and/or sequences covering multiple accessory genes, and further wherein the sequences are at least 1000 bp.

22. The method of claim 21, wherein the biological sample is metagenomic.

23. The method of claim 21, wherein rapid screening is less than 10 minutes.

24. The method of claim 21, wherein the biological sample is substantially free of genomic DNA from the subject.

25. The method of claim 21, wherein the biological sample consists essentially of DNA from one or more biological strains.

26.-27. (canceled)
